(19) United States
(12) Patent Application Publication
(10) Pub. No.: US 2009/0088800 A1
(43) Pub. Date: Apr. 2, 2009

(54) LOOP ROD SPINAL STABLIZATION DEVICE

(75) Inventors: Jason Blain, Encinitas, CA (US); Greg Martin, Encinitas, CA (US); Matt Lake, Encinitas, CA (US)

Correspondence Address: WILSON SONSINI GOODRICH & ROSATI, 650 PAGE MILL ROAD, PALO ALTO, CA, 94304-1050, US

(73) Assignee: SPINAL ELEMENTS, INC., Carlsbad, CA (US)

(21) Appl. No.: 12/195,285

(22) Filed: Aug. 20, 2008

Related U.S. Application Data
(60) Provisional application No. 60/957,986, filed on Aug. 24, 2007.

(51) Int. Cl. A61B 17/70; A61B 17/04

(52) U.S. Cl. 606/246; 606/264; 606/301; 606/278; 606/308

(57) ABSTRACT

A rod-fastener combination device for spinal stabilization is provided. The device combines a rod and a spinal fastener in a single unit, which allows the rod to move relative to the fastener prior to its being fixed in place, e.g. with a set screw.

### CROSS-REFERENCE

This application claims the benefit of U.S. Provisional Application No. 60/957,986, filed Aug. 24, 2007, which application is incorporated herein by reference.

### BACKGROUND OF THE INVENTION

Surgical spinal stabilization is commonly carried out using a combination of hardware, which generally includes pedicle screws and stabilization rods. Pedicle screws combine a bone fixation device (e.g. a screw) and a coupler. The coupler receives a spine-stabilization rod, which is locked in place within the coupler by means of a set screw. Two or more pedicle screws provide anchor points for the rod. The combination of rod and pedicle screws provide mechanical stabilization of the spine, holding the vertebrae in spatial relationship to one another, and immobilizing the spinal segments that are being fused.

Most screws and couplers are constructed such that the bone fixation device has polyaxial movement relative to the coupler prior to being locked into position. The polyaxial screw assembly allows the rod to be placed at a variety of angles relative to the angle at which the screw is placed in the pedicle. This allows the surgeon flexibility in placement of the pedicle screw, as the angle of the coupler may be changed after the screw has been affixed to bone.

In operation, a surgeon implants two or more pedicle screws into vertebrae so that the couplers of the pedicle screws are generally aligned. The surgeon then installs the rod within the couplers of the pedicle screws. Then the surgeon adds a set screw or other locking device into the couplers and tightens down each set screw to lock the rod in place.

While pedicle screw, rod, and coupler systems have many benefits, there remain challenges and inadequacies, which are addressed by the following disclosure, including the attached drawings.

### SUMMARY OF THE DISCLOSURE

Embodiments described herein address various drawbacks to previously described spinal stabilization devices, which have heretofore been unaddressed. In particular, some embodiments described herein address the need for reducing the number of parts used during spinal stabilization surgery.

The foregoing and further needs are met by embodiments set forth herein, of which at least some provide a device for stabilizing the spine, comprising: a combination of a spinal rod and a bone fastener, wherein the combination is adapted to be inserted into a body as a single unit. In some embodiments, the bone fastener comprises a head and a bone fixation member. In some embodiments, the spinal rod is adapted to hinge about an axis through the head. In some embodiments, the spinal rod is adapted to hinge relative to the bone fixation member of the first bone fastener. In some embodiments, the spinal rod comprises a loop defining an eye, wherein the head of the bone fastener comprises a loop engagement element adapted to receive a portion of the loop. In some embodiments, the device comprises a second bone fastener comprising: a head comprising a slot adapted to receive a portion of the spinal rod; a bone fixation member coupled to the head; and a second securing member, wherein the second securing member is adapted to engage the head of the second bone fastener and is adapted to maintain the spinal rod within the slot of the second bone fastener.

Further embodiments described herein provide a spinal stabilization device comprising: a bone fastener comprising a bone fixation member and a head; a spinal rod, wherein the spinal rod is coupled to the bone fastener prior to implantation of the bone fastener; and a hinging means for hinging the spinal rod relative to the bone fastener, wherein the hinging is about an axis defined by a portion of the head of the bone fastener. In some embodiments, the hinging means comprises: a slot defining a post of the head of the bone fastener; and a loop at an end of the spinal rod having a crosspiece, wherein the crosspiece substantially aligns with the axis, and wherein the loop defines an eye through which the post fits. In some embodiments, the hinging means comprises: a pin substantially aligning with the axis, wherein a first portion of the pin fits at least partially within an eye defined by a loop at an end of the spinal rod, and wherein a second portion of the pin fits at least partially within a first aperture of the head.

In some embodiments, there is provided a spinal rod comprising a loop defining an eye. In some embodiments, the loop is at an end of the rod. In some embodiments, the loop comprises a joint at an end of the rod, a first arm extending from the joint, a second arm extending from the joint and a crosspiece connecting the first arm to the second arm. In some embodiments, the crosspiece comprises a surface having at least one notch therein. In some embodiments, the crosspiece comprises a surface having a plurality of notches therein.

Uses and advantages of the rod-fastener device, spinal stabilization device comprising said rod-fastener device, spinal stabilization rod, bone fastener and methods of using the rod-fastener device, spinal stabilization device comprising said rod-fastener device, spinal stabilization rod, bone fastener include: simplification of surgical procedures; reduction in the number of elements that have to be assembled in vivo during a surgical procedure; improved manufacturing efficiency; and/or improved strength of the spinal stabilization device. Other uses and advantages will be apparent to the person skilled in the art upon consideration of the following detailed description and appended figures.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention described herein are set forth with particularity in the appended claims. A better understanding of the features and advantages of the invention will be obtained by reference to the following detailed description that sets forth illustrative embodiments, in which the principles of the invention are utilized, and the accompanying drawings of which:

FIG. 1A shows a perspective view of an embodiment of a new rod-fastener device, wherein the rod of the device is in a first position.

FIG. 1B shows a side plan view of an embodiment of a rod-fastener device, wherein the rod of the device is in a first position.

FIGS. 2A, 2B and 2C depict three views of a new rod-fastener device. The rod of the device has been rotated about an axis x from the first position (shown in FIGS. 1A and 1B) to a second position.

FIG. 3A shows a perspective view of an embodiment of a new rod-fastener device, wherein the rod of the device is in a first position.

FIG. 3B depicts a perspective view of the rod-fastener device shown in FIG. 3A; the rod of the device has been rotated about an axis x from the first position (shown in FIG. 3A) to a second position.

FIGS. 4A-4D depict various views of a new spinal rod that is adapted for use in a novel rod-fastener as described herein.

FIGS. 5A-5C show a side view of a bone fastener adapted for use in a new rod-fastener as described herein.

FIGS. 6A-6C show perspective, side plan and top-down views, respectively, of a securing member adapted for use in a new rod-fastener as described herein.

FIG. 7 shows an alternative embodiment of a new rod-fastener as described herein.

FIGS. 8A and 8B show two views of an alternative embodiment of a new rod-fastener as described herein.

FIG. 9A shows a side view of an alternative embodiment of a rod-fastener as described herein, wherein the rod is in a first position.

FIG. 9B shows a side view of an alternative embodiment of a rod-fastener as described herein, wherein the rod has been rotated about axis x.

### DETAILED DESCRIPTION

Provided herein is a device for stabilizing a spine, comprising: a combination of a spinal rod and a first bone fastener, wherein the combination is adapted to be inserted into a body as a single unit. In some embodiments, the first bone fastener comprises a head and a bone fixation member. In some embodiments, the spinal rod is adapted to hinge about an axis through the head. In some embodiments, the spinal rod is adapted to hinge relative to the bone fixation member of the first bone fastener.

Some embodiments provide a device of paragraph [0025], wherein the bone fastener further comprises a first securing member. In some embodiments, the first securing member is adapted to lock the spinal rod relative to the first bone fastener. In some embodiments, the first securing member allows limited hinging of the spinal rod about the axis relative to the first bone fastener while securing the general position of the of the spinal rod relative to the first bone fastener. In some embodiments, the first securing member is adapted to engage the head and is adapted to engage the spinal rod. In some embodiments, the first securing member is a set screw. In some embodiments, the set screw comprises a head engagement member. In some embodiments, the bead engagement member comprises external threads. In some embodiments, the head comprises internal threads. In some embodiments, the external threads of the head engagement member are adapted to engage the internal threads of the bead to resist proximal motion of the set screw. In some embodiments, the external threads of the head engagement member are adapted to engage the internal threads of the head and wherein the external threads and internal threads are adapted to convert rotational force of turning the set screw about an axis defined by the bone fastener into distally-directed force. In some embodiments, a distal surface of the set screw is adapted to engage a surface of the spinal rod, whereby turning the set screw imparts distal force to the spinal rod. In some embodiments, the first securing member is a nut. In some embodiments, the nut comprises a head engagement member. In some embodiments, the head comprises an overhang. In some embodiments, the head engagement member of the nut is adapted to engage the overhang of the head. In some embodiments, the head engagement member is adapted to engage the overhang to resist proximal motion of the nut. In some embodiments, the head engagement member is adapted to engage the overhang of the head and wherein the head engagement member and the overhang are adapted to convert rotational force of turning the nut about an axis defined by the bone fastener into distally-directed force. In some embodiments, the overhang comprises an inclined surface. In some embodiments, the overhang is on at least one of an inner surface of the head and an outer surface of the head. In some embodiments, the head engagement member is adapted to engage at least one of an inner surface of the head and an outer surface of the head. In some embodiments, the nut comprises a cam surface. In some embodiments, the cam surface is on the head engagement member. In some embodiments, distal surface of the nut is adapted to engage a surface of the spinal rod, whereby turning the nut imparts distal force to the spinal rod. In some embodiments, the cam surface is on a distal surface of the nut. In some embodiments, the cam surface is adapted to engage a surface of the spinal rod, whereby turning the nut imparts distal force to the spinal rod.

Some embodiments provide a device of one of paragraphs [0025]-[0026], wherein the head and the bone fixation member together form an integral unit. In some embodiments, the head and the bone fixation member together form a monoaxial bone fastener. In some embodiments, the monoaxial bone fastener is a unitary device cast into, or machined from, a single piece of metal.

Some embodiments provide a device of one of paragraphs [0025]-[0027] wherein the head has a distal end comprising an aperture through which the bone fixation member passes. In some embodiments, the head and the bone fixation member form a polyaxial bone fastener.

The device of one of paragraphs [0025]-[0028] wherein the spinal rod comprises a loop defining an eye, wherein the head of the first bone fastener comprises a loop engagement element adapted to receive a portion of the loop. In some embodiments, the loop engagement element comprises a slot. In some embodiments, the spinal rod comprises a pin adapted to be inserted through the eye of the loop, along the axis, and through a first aperture of the head of the first bone fastener. In some embodiments, the pin is adapted to be inserted through a second aperture of the head of the first bone fastener. In some embodiments, the pin is adapted to maintain the portion of the loop coupled to the loop engagement element. In some embodiments, the loop defining the eye is formed in an end of the spinal rod. In some embodiments, the loop comprises a joint connected to an end of the spinal rod, a first arm projecting from the joint, a second arm projecting from the joint and a crosspiece connecting the first arm to the second arm. In some embodiments, the crosspiece comprises a surface having at least one notch therein. In some embodiments, the crosspiece comprises a surface having a plurality of notches therein. In some embodiments, the plurality of notches are substantially parallel or form a crisscross pattern. In some embodiments, the loop defining the eye is formed in a medial portion of the spinal rod. In some embodiments, the slot defines a post and a pillar (which may also be called a first post and a second post). In some embodiments, at least a portion of the post fits within the eye. In some embodiments, at least one of the post and the pillar comprises a securing member engagement element. In some embodiments, the first securing member is adapted to engage the securing member engagement element to resist proximal motion of the first securing member. In some embodiments, the spinal rod comprises a bar, and wherein the slot of the head is adapted to receive a portion of the bar. In some embodiments, a distal portion of the first securing member is adapted to engage at least one of a surface of the loop and a surface of the bar, whereby turning the first securing member imparts distal force to at least one of the surface of the loop and the surface of the bar.

Some embodiments provide a device of one of paragraphs [0025]-[0029], wherein the head of the first bone fastener comprises a loop defining an eye, and wherein the spinal rod comprises a loop engagement element adapted to receive a portion of the loop. In some embodiments, the loop engagement element comprises a slot. In some embodiments, the spinal rod comprises a pin adapted to be inserted through the eye of the loop, along the axis, and through a first aperture of the spinal rod. In some embodiments, the pin is adapted to be inserted through a second aperture of the spinal rod. In some embodiments, the pin is adapted to maintain the portion of the loop coupled to the loop engagement element. In some embodiments, the loop defining the eye is formed in an end of the spinal rod.

Some embodiments provide a device of one of paragraphs [0025]-[0030], further comprising a second bone fastener comprising: a head comprising a slot adapted to receive a portion of the spinal rod; a bone fixation member coupled to the head; and a second securing member, wherein the second securing member is adapted to engage the head of the second bone fastener and is adapted to maintain the spinal rod within the slot of the second bone fastener. In some embodiments, the second securing member is adapted to lock the spinal rod relative to the second bone fastener. In some embodiments, the second securing member adapted to engage the head of the second bone fastener is a set screw. In some embodiments, the set screw comprises a head engagement member. In some embodiments, the head engagement member comprises external threads. In some embodiments, the head comprises internal threads. In some embodiments, the external threads of the engagement member are adapted to engage the internal threads of the head to resist proximal motion of the nut. In some embodiments, the external threads of the engagement member are adapted to engage the internal threads of the head and wherein the external threads and internal threads are adapted to convert rotational force of turning the set screw about an axis defined by the bone fastener into distally-directed force. Some embodiments provide a distal surface of the set screw is adapted to engage the portion of the spinal rod, whereby turning the set screw imparts distal force to the portion of the spinal rod. In some embodiments, the second securing member adapted to engage the head of the second bone fastener is a nut. In some embodiments, the comprises a head engagement member. In some embodiments, the head comprises an overhang. In some embodiments, the head engagement member of the nut is adapted to engage the overhang of the head. In some embodiments, the head engagement member is adapted to engage the overhang to resist proximal motion of the nut. In some embodiments, the head engagement member is adapted to engage the overhang of the head and wherein the head engagement member and the overhang are adapted to convert rotational force of turning the nut about an axis defined by the bone fastener into distally-directed force.

Some embodiments provide a device of one of paragraphs [0025]-[0031], wherein the overhang comprises an inclined surface. In some embodiments, the overhang is on at least one of an inner surface of the head and an outer surface of the head. In some embodiments, the head engagement member is adapted to engage at least one of an inner surface of the head and an outer surface of the head. In some embodiments, the nut comprises a cam surface. In some embodiments, the cam surface is on the head engagement member. In some embodiments, a distal surface of the nut is adapted to engage the portion of the spinal rod, whereby turning the nut imparts distal force to the portion of the spinal rod. In some embodiments, the cam surface is on a distal surface of the nut. In some embodiments, the cam surface is adapted to engage a surface of the spinal rod, whereby turning the nut imparts distal force to the spinal rod. In some embodiments, the head and the bone fixation member of the second bone fastener together form an integral unit. In some embodiments, the head and the bone fixation member of the second bone fastener together form a monoaxial bone fastener. In some embodiments, the head of the second bone fastener has a distal end comprising an aperture through which the bone fastener passes. In some embodiments, the head and the bone fixation member together form a polyaxial bone fastener.

Some embodiments provide a method of using a device comprising: fastening a bone fixation member of a first bone fastener to a first pedicle of a first vertebra, wherein the first bone fastener is coupled to a spinal rod prior to the step of fastening; fastening a bone fixation member of a second bone fastener to a second pedicle of a second vertebra, wherein the second bone fastener comprises a head having a slot adapted to receive a portion of the spinal rod; hinging the rod about an axis defined by a head of the first bone fastener toward the second bone fastener; placing the portion of the spinal rod into the slot of the second bone fastener, and engaging a second securing member of the second bone fastener with the head of the second bone fastener, whereby the second securing member maintains the spinal rod within the slot of the second bone fastener. In some embodiments, the method employs a device of one of paragraphs [0025]-[0032]. In some embodiments the method further comprises: engaging a first securing member of the first bone fastener with the head of the first bone fastener. In some embodiments, the first securing member locks the spinal rod relative to the first spinal fixation device. In some embodiments, the first securing member of the first bone fastener with the head of the first bone fastener allows limited hinging of the spinal rod relative to the first bone fastener while securing the general position of the of the spinal rod relative to the first bone fastener.

Some embodiments provide a spinal rod comprising a loop defining an eye. In some embodiments, the spinal rod is adapted for use in a device of one of paragraphs [0025]-[0032] or a method of paragraph [0033]. In some embodiments, the loop is at an end of the rod. In some embodiments, the loop comprises a joint at an end of the rod, a first arm extending from the joint, a second arm extending from the joint and a crosspiece connecting the first arm to the second arm. In some embodiments, the crosspiece comprises a surface having at least one notch therein. In some embodiments, the crosspiece comprises a surface having a plurality of notches therein. In some embodiments, the plurality of notches are substantially parallel or form a crisscross pattern. In some embodiments, the loop is at a medial portion of the rod. In some embodiments, at least a portion of the post of a spinal stabilization device of one of paragraphs [0025]-[0032] fits within the eye. In some embodiments, the spinal stabilization device comprises a pin adapted to be inserted through the loop.

Some embodiments provide a spinal rod comprising a slot and a first aperture at an end of the spinal rod. In some embodiments, the rod further comprises a pin adapted to be inserted through the first aperture. In some embodiments, the rod further comprises a second aperture across the slot from the first aperture. In some embodiments, the rod further comprises a pin adapted to be inserted across the slot and adapted to be inserted through at least one of the first aperture and the second aperture.

Some embodiments provide a spinal stabilization device comprising: a bone fastener comprising a bone fixation member and a head; a spinal rod, wherein the spinal rod is coupled to the bone fastener prior to implantation of the bone fastener; and a hinging means for hinging the spinal rod relative to the bone fastener, wherein the hinging is about an axis defined by a portion of the head of the bone fastener. In some embodiments, the hinging means comprises: a slot defining a post of the head of the bone fastener; and a loop at an end of the spinal rod having a crosspiece, wherein the crosspiece substantially aligns with the axis, and wherein the loop defines an eye through which the post fits. In some embodiments, the hinging means comprises: a pin substantially aligning with the axis, wherein a first portion of the pin fits at least partially within an eye defined by a loop at an end of the spinal rod, and wherein a second portion of the pin fits at least partially within a first aperture of the head. In some embodiments, the hinging means comprises: a pin substantially aligning with the axis, wherein the pin comprises a loop, wherein a first portion of the pin fits at least partially within a first aperture of the head, and wherein at least a portion of the spinal rod fits within the loop of the pin.

Thus, provided herein is a spinal stabilization device comprising a rod-fastener assembly. The unitary rod-fastener forms part of a spinal stabilization device. In some embodiments, the rod-fastener is implanted as a unit, the fastener (which is e.g. a spinal screw or barbed nail) forming one end or the other of a spinal stabilization device. One or more pedicle screws can then be attached to one or more vertebrae and the rod urged into place within the additional pedicle screw or screws. Thus, in at least some embodiments, the rod-fastener reduces the number of parts that must be assembled in order to stabilize a spine, which thereby reduces the complexity, duration or both of the surgery.

Further understanding of the present disclosure may be gained upon consideration of, and reference to, the following description of the drawings, which are illustrative and non-limiting.

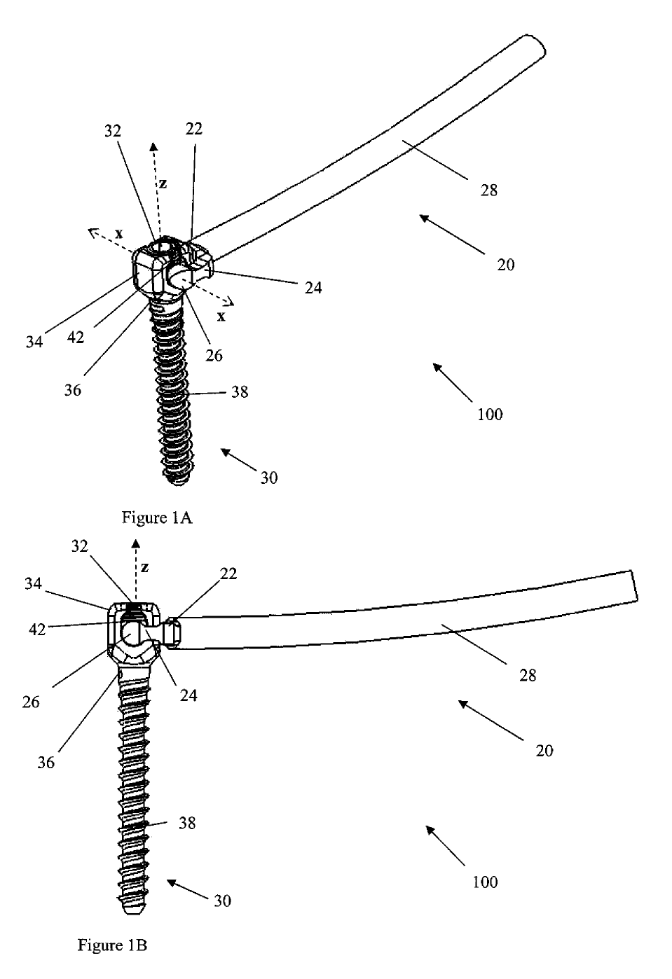

FIGS. 1A and 1B depict perspective and side-plan views of an embodiment of a rod-fastener 100 disclosed herein. A rod-fastener 100 comprises a rod 20 and a fastener 30. In the depicted embodiment, the fastener 30 is a screw comprising a bone fixation member that is a threaded shank 38 and a head 34. However, the fastener 30 can comprise any type of bone fixation member, such as a nail or staple, provided that the bone fixation member is adapted to be inserted into a bone and held in place with sufficient strength and durability to form a lasting anchor for the spinal stabilization device. The fastener 30 also comprises a head 34, which is connected to the shank 38 at a junction 36.

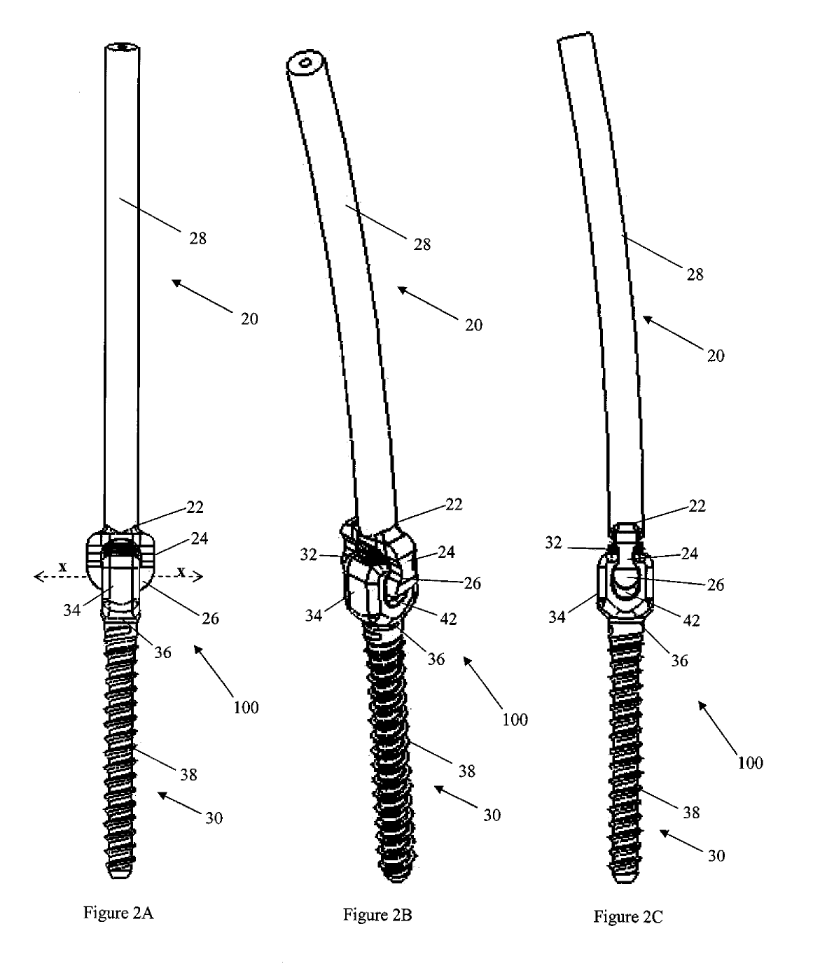

The rod 20 comprises arms 24, which are connected to the rod shaft 28 at a joint 22. A cross member 26 connects the arms 24 and passes through the head 34 of the fastener 30 through a slot 42. A securing member 32 is inserted into the slot 42 to hold the cross member 26 in the slot 42, thereby maintaining the fastener 30 and the rod 20 in an assembled state. As show in FIGS. 1A-1B and 2A-2C, the rod-screw 100 is capable of adopting plural configurations, as the cross member 26 and the slot 42 in the head 34 form a hinge about which the rod 20 may be hinged (about an axis x) to a variety of positions, of which two are depicted in FIGS. 1A-1B and 2A-2C, respectively. The depicted securing member 32 is a set screw that may be locked into the head 34 via an overhang (not depicted). The securing member 32 may be screwed toward the crosspiece 26 of the rod 20 by turning the securing member 32 about the z axis, which passes through the center of the securing member 32.

In the embodiment depicted in FIGS. 1A-1B, the head 34 and the shank 38 are formed together during manufacturing as an integral unit; however it will be recognized that the head 34 and shank 38 can be formed separately and assembled at some time prior to insertion of the shank 38 into a body. For example, in some embodiments, the head and shank may be manufactured as separate units, which may be assembled prior to implantation. In some embodiments, the head may have an aperture through which the shank (fixation member) of the fastener is inserted, and through which it projects, thereby providing a functional fastener unit.

As depicted in FIGS. 1A and 1B the rod-fastener 100 may be pre-assembled prior to implantation of the rod-fastener. The rod 20 and fastener 30 (bone fastener) may be made of suitable biologically inert materials with suitable strength and flexibility. In some embodiments, suitable materials are biologically inert metals such as surgical steel and titanium.

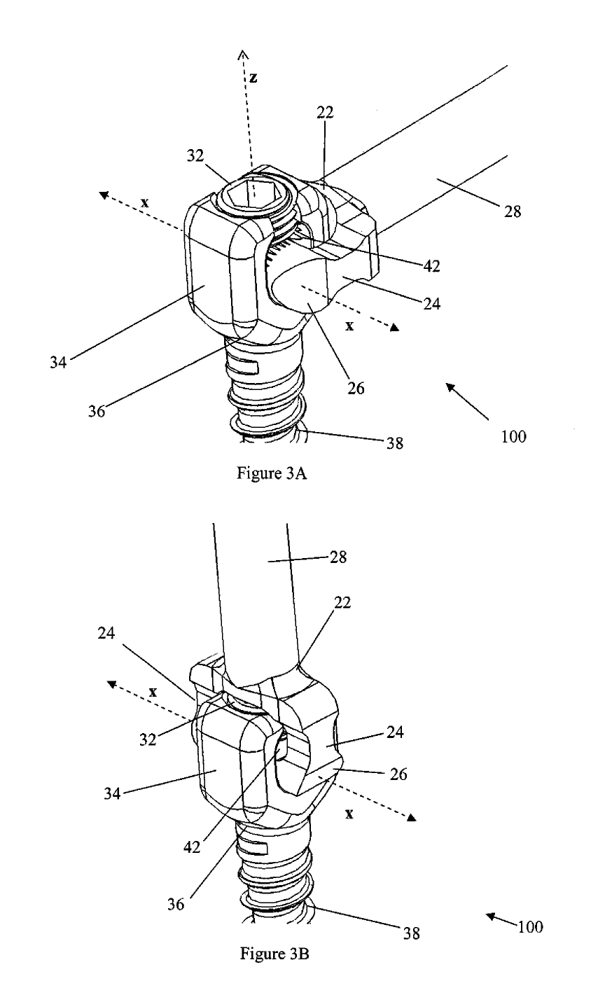

FIGS. 3A and 3B show expanded views of the hinging portion of the rod-fastener 100 as depicted in FIGS. 1A-1B and 2A-2C.

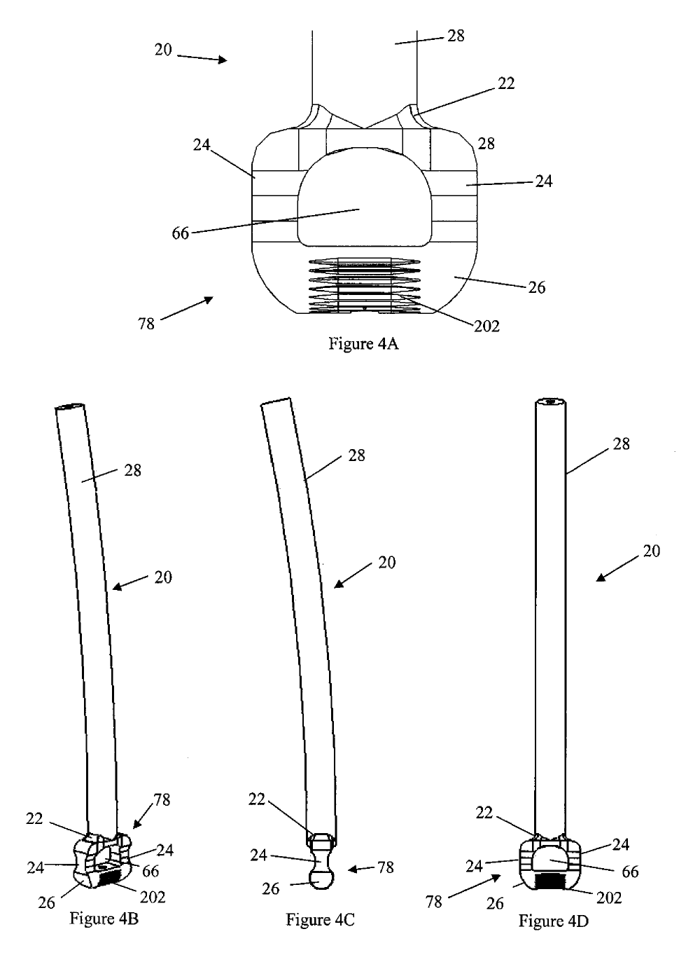

FIGS. 4A-4D depict various views of an embodiment of a rod 20 that may be employed in manufacturing a rod-fastener as described herein. The rod 20 comprises a shaft 28, which terminates in a loop 78. The loop 78 is formed of two arms 24, which are joined in some embodiments by a crosspiece 26. The two arms 24 are connected to the shaft 28 of the arm 20 by a joint 22. Together the joint 22, arms 24 and crosspiece 26 form a loop 78, which defines an eye 66. In the depicted embodiment, the crosspiece 26 comprises a plurality of indentations 202, which may serve to provide traction for a securing member.

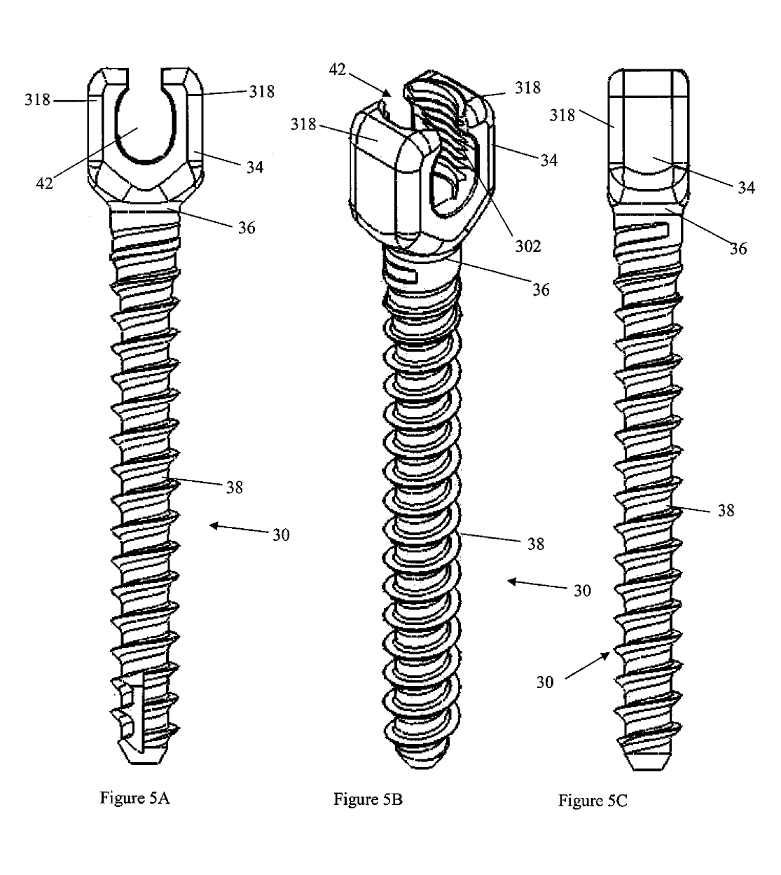

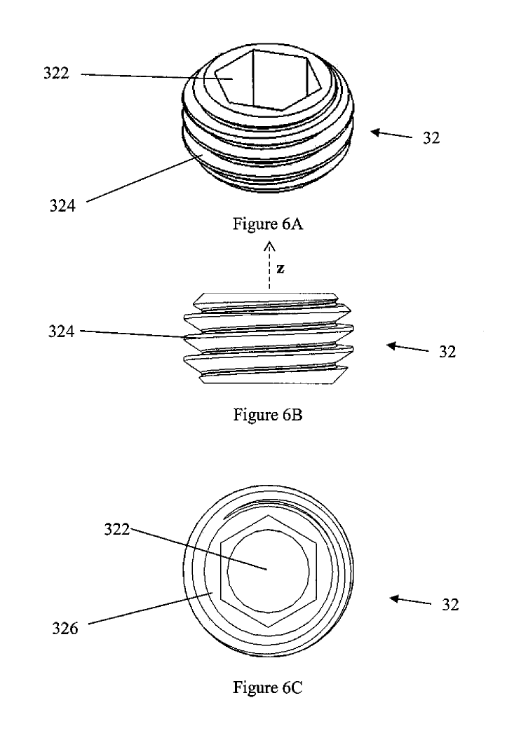

FIGS. 5A-5C depict various views of a fastener 30 (bone fastener) that may be employed in manufacturing a rod-fastener as described herein. The fastener 30 comprises a head 34 and a threaded shaft 38, which are joined to one another by a junction 36. In the depicted embodiment, the fastener 30 is a screw that is advantageously manufactured as a single unit. The head 34 has a pair of posts 318 (also called a post and a pillar, or a first post and a second post), which define a slot 42. The posts 318 each have a threaded inner surface 302, which is adapted to engage a securing member 32 as described in e.g. FIG. 1A and FIGS. 6A-6C. The head of the bone fastener may include a loop engagement element, which may include at least one of: a post (a first post), a second post (a pillar), and a slot, among other elements, and is configured to (or adapted to) receive at least a portion of the loop of the rod. The slot may define the posts, the first post and the second post, and/or a post and a pillar. At least a portion of a post or a pillar fits within the eye of the rod.

FIGS. 6A-6C depict various views of a securing member 32. In the depicted embodiment, the securing member 32 is a set screw comprising a threaded shaft 324 that is rotatable about an axis z by engaging the hex key slot 322 with a suitably shaped driving tool, such as a hex key (Allen wrench). It will be apparent to the person skilled in the art that though the securing member 32 is depicted with a hexagonal indentation for receiving a driving tool, other shapes are possible. Among the available shapes are single slots, crossed (Philips-type) slots, and a variety of stars and other configurations. In addition, though the threaded inner surface 302 of the head 34 and the threaded shaft 324 of the securing member 32 are depicted as being threaded with several threads, they may actually form opposing cammed surfaces that engage and allow tightening of the securing member 32 by a single turn about the axis z or less.

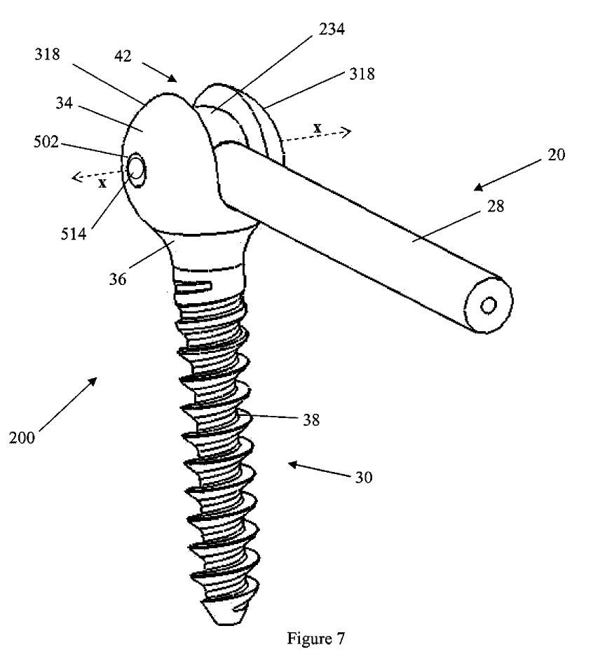

FIG. 7 depicts an alternative embodiment of a rod-fastener 200, which is described herein. The rod-fastener 200 comprises a rod 20 and a fastener 30. The screw (fastener) 30 comprises a threaded shank 38 and a slotted head 34. The head 34 is connected to the threaded shank 38 by a joint 36. The head 34 comprises two posts 318 (which in the depicted embodiment are rounded, but my also be more angular in configuration), which define a slot 42. The rod 20 comprises a shaft 28 and a cap 234, which fits inside the slot 42 and is connected to the shaft 28. A connector pin 514 acts as a securing member, passing through a hole 502 in each of the posts 318 and the cap 234 of the rod 20, thereby connecting it to the fastener 30 and allowing the rod 20 to pivot or hinge with respect to the fastener 30.

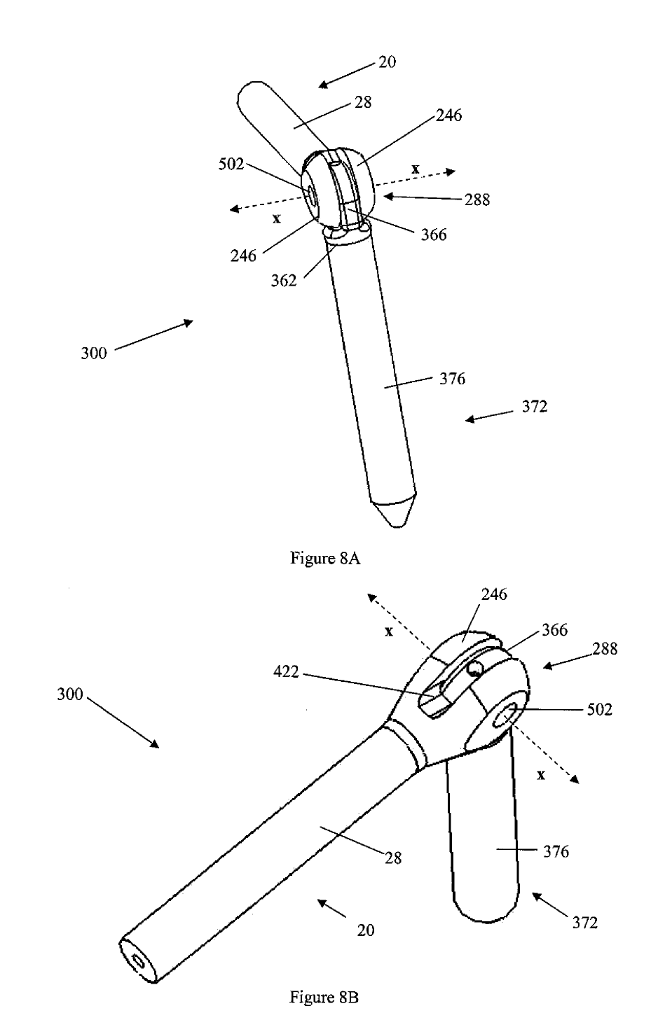

FIGS. 8A-8B depict two different views of an alternative embodiment of a rod-fastener 300. The rod-fastener 300 comprises a rod 20 and a nail 372. The nail 372 comprises a shank 376 and a post 366. The post 366 is connected to the shank 376 by a joint 362. The rod 20 comprises a shaft 28 and a cap 288, which comprises two arms 246 (which in the depicted embodiment are rounded to accommodate the hinging motion of the rod 20), which define a slot 422. The post 366 fits between the two arms 246. A connector pin (not shown in this view but analogous to the pin 514 in FIG. 7) acts as a securing member, passing through a hole 502 in each of the arms 246 and the post 366 to connect the rod 20 to the fastener 372 and allowing the rod 20 to pivot or hinge with respect to the fastener 372.

The embodiment of FIGS. 8A and 8B may also be described as having a spinal rod 20 comprising a loop (or an arm 246) that defines an eye (the hole 502 in the arm 246). The head of the bone fastener (the nail 372) comprises a loop engagement element (the post 366) adapted to receive a portion of the loop (a side of the arm 246, and/or a pin). The spinal rod may also comprise a pin (not shown in this view but analogous to the pin 514 in FIG. 7) adapted to be inserted through the eye (the hole 502 in the arm 246) of the loop along an axis (depicted by dotted lines x) defined by the eye (or the hole 502 in the arm 246), and through a first aperture (a hole in the post 366) of the head of the bone fastener. The pin connects the rod to the fastener. The pin is adapted to maintain the portion of the loop coupled to the loop engagement element.

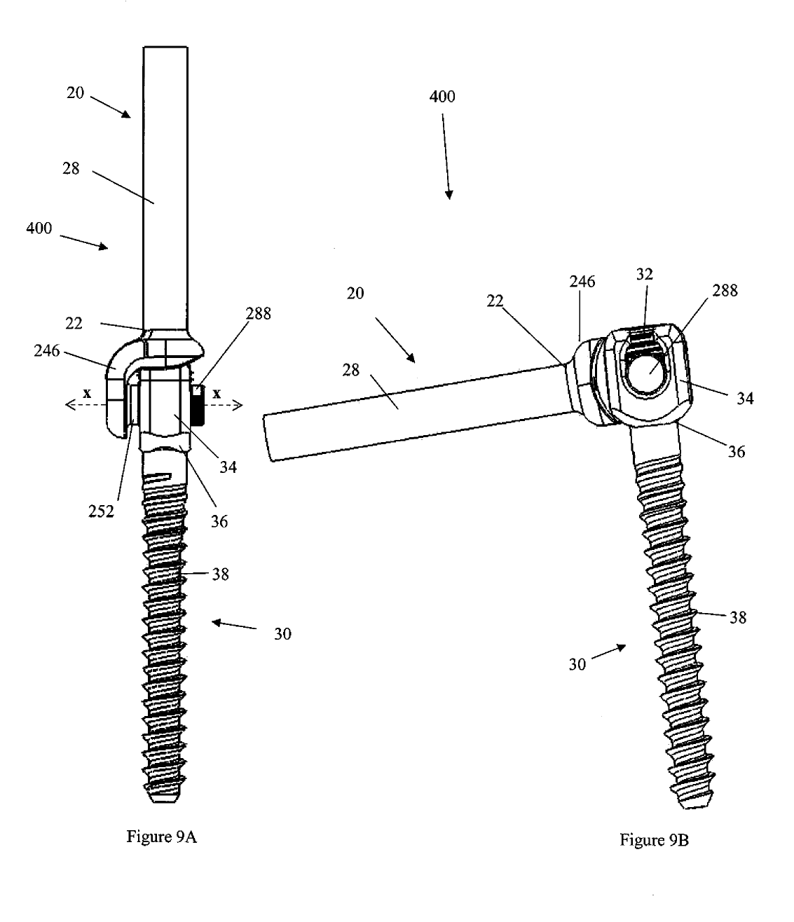

FIGS. 9A and 9B depict an alternative embodiment of a rod-fastener 400, which is described for the first time herein. A rod-fastener 400 comprises a rod 20 and a fastener 30. In the depicted embodiment, the fastener 30 is a screw comprising a threaded shank 38 and a head 34, which is connected to the shank 38 at a junction 36. The rod 20 comprises arm 246, which is connected to the rod shaft 28 at a joint 22. A pivot member 252 (or pin) passes through an aperture in the arm 246 and through the head 34. A pivot securing member 288 caps the pivot member 252 and prevents the head 34 from slipping off the pivot member 252, thereby holding the rod 20 and the fastener 30 together. As show in FIGS. 9A-9B, the rod-fastener 400 is capable of adopting plural configurations, as the rod 20 may pivot about the pivot member 252 and about the x axis, which passes through the center of the pivot member 252. Thus, in some embodiments, the spinal stabilization device comprises a spinal rod comprising a pin (e.g. pivot member 252) adapted to be inserted through an aperture in the rod 20 and through an eye of the loop in the head 34 of the fastener 30. The loop of the may be a complete loop (i.e. oval or O-shaped, for example) or incomplete loop (i.e. may be C-shaped, for example), as shown in FIGS. 9A and 9B, as it may be adapted to receive a securing member as described elsewhere herein (such as a set screw or a nut, for non-limiting example) and shown in FIG. 9B.

### Method of Using a Spinal Stabilization Device as Described Herein

A rod-fastener (rod-screw) as described herein may be employed in method of stabilizing a spine. The rod-fastener as described herein, e.g. in FIGS. 1A-1B, 2A-2C, 3A-3B, 7, 8A-8B or 9A-9B, is implanted e.g. by straightening the rod-fastener out so the shank of the fastener and the shaft of the rod lie on substantially the same axis. The bone fixation member (screw or nail) of the fastener is then turned (screwed) or impacted (hammered) into a pedicle such that the rod, when turned about the hinge formed between the rod and the fastener, is in alignment with the spine. One or more, preferably two or more pedicle screws are inserted into one or more, preferably two or more vertebrae, and the rod is urged into juxtaposition with them. The rod is then locked in contact with the additional pedicle screws. Finally, the securing member is locked in place, e.g. by turning it about the axis that it has in common with the fastener shank. Thus the fastener of the rod-fastener forms a terminal anchor point for the spinal stabilizer.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A device for stabilizing the spine, comprising:
a combination of a spinal rod and at least a first bone fastener,
wherein the combination is adapted to be inserted into a body as a single unit,
and wherein the first bone fastener comprises a head and a bone fixation member.

2. The device of claim 1, wherein the spinal rod is adapted to hinge about an axis through the head of the bone fastener.

3. The device of claim 1, and wherein the spinal rod is adapted to hinge relative to the bone fixation member of the bone fixation fastener.

4. The device of claim 1, wherein the first bone fastener further comprises a first securing member adapted to lock the spinal rod relative to the first bone fastener.

5. The device of claim 1, wherein the first bone fastener further comprises a first securing member operable to allow limited hinging of the spinal rod about the axis relative to the first bone fastener while securing the general position of the of the spinal rod relative to the bone fastener.

6. The device of one of claims 4 and 5, wherein the first securing member is a set screw.

7. The device of claim 6, wherein external threads of the set screw are adapted to engage internal threads of the head to at least one of: resist proximal motion of the set screw, and to convert rotational force of turning the set screw about an axis defined by the bone fastener into distally-directed force.

8. The device of claim 4, wherein the first securing member is a nut.

9. The device of claim 8, wherein the nut comprises a head engagement member, wherein the head comprises an overhang, and wherein the head engagement member of the nut is adapted to engage the overhang of the head in order to at least one of: resist proximal motion of the nut, and convert rotational force of turning the nut about an axis defined by the bone fastener into distally-directed force.

10. The device of claim 9 wherein the overhang of the head comprises an inclined surface.

11. The device of claim 9, wherein the overhang is on at least one of an inner surface of the head and an outer surface of the head.

12. The device of claim 9 wherein the nut comprises a cam surface on the head engagement member.

13. The device of claim 12, wherein a distal surface of the nut is adapted to engage a surface of the spinal rod, whereby turning the nut imparts distal force to the spinal rod.

14. The device of claim 12, wherein the cam surface is on a distal surface of the nut.

15. The device of claim 1, wherein the head and the bone fixation member form a polyaxial bone fastener.

16. The device of claim 1,
wherein the spinal rod comprises a loop defining an eye,
wherein the head of the first bone fastener comprises a loop engagement element adapted to receive a portion of the loop.

17. The device of claim 16, wherein the head comprises a securing member engagement element.

18. The device of claim 17, wherein a first securing member is adapted to engage the securing member engagement element to at least one of: resist proximal motion of the first securing member, impart distal force to the loop, and impart distal force to the rod.

19. The device of claim 17, wherein the loop engagement element comprises a slot that defines a first post and a second post, and wherein at least one of the first post and the second post comprises the securing member engagement element.

20. The device of one of claims 16, 17, and 18, wherein the spinal rod comprises a pin adapted to be inserted through the eye of the loop, along an axis defined by the eye, and through at least one of a first aperture of the head of the first bone fastener and a second aperture of the head of the first bone fastener.

21. The device of claim 1,
wherein the head of the first bone fastener comprises a loop defining an eye, and
wherein the spinal rod comprises a loop engagement element adapted to receive a portion of the loop.

22. The device of claim 21, wherein a pin is adapted to maintain the loop coupled to the loop engagement element.

23. The device of claim 1, further comprising a second bone fastener comprising:
a head comprising a slot adapted to receive a portion of the spinal rod;
a bone fixation member coupled to the head; and
a second securing member,
wherein the second securing member is adapted to engage the head of the second bone fastener and is adapted to maintain the spinal rod within the slot of the second bone fastener.

24. A spinal stabilization device comprising:
a bone fastener comprising a bone fixation member and a head;
a spinal rod, wherein the spinal rod is coupled to the bone fastener prior to implantation of the bone fastener; and
a hinging means for hinging the spinal rod relative to the bone fastener, wherein the hinging is about an axis defined by a portion of the head of the bone fastener.

25. The spinal stabilization device of claim 24, wherein the hinging means comprises:
a slot defining a post of the head of the bone fastener; and
a loop at an end of the spinal rod having a crosspiece,
wherein the crosspiece substantially aligns with the axis, and
wherein the loop defines an eye through which the post fits.

26. The spinal stabilization device of claim 24, wherein the hinging means comprises:
a pin substantially aligning with the axis,
wherein a first portion of the pin fits at least partially within an eye defined by a loop at an end of the spinal rod, and wherein a second portion of the pin fits at least partially within a first aperture of the head.

27. The spinal stabilization device of claim 24, wherein the hinging means comprises:
a pin substantially aligning with the axis,
wherein the pin comprises a loop,
wherein a first portion of the pin fits at least partially within a first aperture of the head, and
wherein at least a portion of the spinal rod fits within the loop of the pin.

28. A method of using a device for stabilizing a spine, comprising:
fastening a bone fixation member of a first bone fastener to a first pedicle of a first vertebra, whereby the first bone fastener is coupled to a spinal rod prior to the step of fastening;
fastening a second bone fixation member of a second bone fastener to a second pedicle of a second vertebra, wherein the second bone fastener comprises a head having a slot adapted to receive a portion of the spinal rod;
hinging the rod about an axis defined by a head of the first bone fastener toward the second bone fastener;
placing the portion of the spinal rod into the slot of the second bone fastener, and engaging a second securing member of the second bone fastener with the head of the second bone fastener, whereby the second securing member maintains the spinal rod within the slot of the second bone fastener.

29. The method of claim 28, further comprising:
engaging a first securing member of the first bone fastener with the head of the first bone fastener.

30. The method of claim 29, whereby the first securing member locks the spinal rod relative to the first spinal fixation device.

31. The method of claim 29, whereby engaging the first securing member of the first bone fastener with the head of the first bone fastener allows limited hinging of the spinal rod relative to the first bone fastener while securing the general position of the of the spinal rod relative to the first bone fastener.

32. The method of claim 28, wherein the device is one of claims 1-27.

33. A spinal rod comprising a loop defining an eye.

34. The spinal rod of claim 33, wherein the loop is at an end of the rod.

35. The spinal rod of claim 33, wherein at least a portion of a first post or second post of a spinal stabilization device of claim 19 fits within the eye.

36. A spinal rod comprising a slot and a first aperture at an end of the spinal rod.

37. The spinal rod of claim 36, further comprising a pin adapted to be inserted through the first aperture.

38. The spinal rod of claim 36, further comprising a second aperture across the slot from the first aperture.

39. The spinal rod of claim 38, further comprising a pin adapted to be inserted across the slot and adapted to be inserted through at least one of the first aperture and the second aperture.
